(19) United States
(12) Patent Application Publication
(10) Pub. No.: US 2024/0101595 A1
(43) Pub. Date: Mar. 28, 2024

(54) SYSTEMS AND METHODS FOR MULTI-REAGENT CHEMICAL LABELING OF BIOLOGICAL MOLECULES

(71) Applicant: Case Western Reserve University, Cleveland, OH (US)

(72) Inventors: Mark R. Chance, Cleveland, OH (US); Janna Kiselar, Cleveland, OH (US)

(21) Appl. No.: 18/264,161

(22) PCT Filed: Feb. 4, 2022
(86) PCT No.: PCT/US22/15193

§ 371 Date: Aug. 3, 2023

Related U.S. Application Data
(60) Provisional application No. 63/145,851, filed on Feb. 4, 2021.

(51) Int. Cl. C07K 1/13; G01N 33/58; G01N 33/68

(52) U.S. Cl. CPC C07K 1/13; G01N 33/58; G01N 33/6848

(57) ABSTRACT

A one-pot method of multiple protein labeling that is suitable for protein footprinting is disclosed, along with systems, auxiliary devices, kits, and non-transitory computer readable mediums relating to the same. Hydroxyl radicals are maintained at a concentration for a length of time sufficient to both react with trifluoromethyl precursors and a protein of interest, where the reaction with the trifluoromethyl precursors further maintains a trifluoromethyl concentration for a length of time sufficient to react with the protein of interest. The multiplex labeling achieves significant improvements over the state of the art labeling methods, both by increasing the types of amino acids which can be labeled in a single experiment and by increasing the overall labeling coverage.

### CROSS-REFERENCE TO RELATED APPLICATIONS

This application is related to, claims priority to, and incorporates herein by reference for all purposes U.S. Provisional Patent Application No. 63/145,851, filed Feb. 4, 2021.

### STATEMENT REGARDING FEDERALLY FUNDED RESEARCH

Not applicable.

### BACKGROUND

The field of protein footprinting using covalent labeling was spearheaded by the inventors, among others, over the past roughly two decades of its existence. It was developed using ideas from hydrogen-deuterium exchange mass spectrometry, whose value in examining protein structure and dynamics clearly proved the value of the basic “bottom up” proteomics (examination of proteins at the peptide level) conceptual approach. The first protein footprinting using irreversible (covalent) labeling was achieved with hydroxyl radicals generated from a synchrotron x-ray source around the turn of the century, achieving modifications in microsecond-millisecond timescales. Sharp and Hettich and Gross and colleagues at Washington University expanded the technique with the introduction of photochemical oxidation of proteins and then microsecond-millisecond footprinting using laser initiation (FPOP), where peroxide precursors are utilized with optical sources to generate the hydroxyl radicals. It has been consistently demonstrated that rapid covalent modification of target proteins (fast footprinting) is advantageous from the standpoints of: promoting the primary radical-target reaction, disfavoring radical-radical side reactions, overcoming scavenging of reagents interfering with the experiment, and enhancing overall signal-to noise of the subsequent mass spectrometry analysis.

The technique from the inventors was refined and software was released around 2009 to automate the protein footprinting analysis. Recently, researchers at the University of Wisconsin-Madison have further extended the technique to tabletop plasma generation of radicals. Throughout, the technique has been limited by a few factors, detailed below.

First, there are a limited number of chemistries that have been shown to work with protein footprinting. The most prominent is the aforementioned hydroxyl radical footprinting. Beyond that, there are only three other widely used chemistries, each having significant limitations. Carboxyl groups can be modified by being activated with 1-ethyl-3-(3-dimethylaminopropyl)-carbodiimide (EDC), followed by quenching of the activated species by glycine-ethyl-ester (GEE). EDC/GEE modification only targets two residues, e.g. representing 9-10% of typical protein sequence. Methylene carbine chemistry was capable of labeling residues with methylene, but achieved minimal specificity and required utilizing photolysis of diazirine-related compounds. Carbethoxy labeling was achieved using diethyl-pyrocarbonate reaction with side-chain nucleophiles. The carbethoxy chemistry only labels ˜6 out of 20 amino acids.

Second, the existing chemistries, even for hydroxyl radical labeling do not achieve labeling for greater than 10-12 of the 20 amino acids most regularly found in proteins in a single experiment.

Third, due to dynamic range issues of both modification and detection, the existing chemistries provide only modest coverage for the individual residues in the detected peptides derived from the protein. Hydroxyl radical chemistry achieves ˜5-15% of residues detected as being labeled “total” across the protein, rarely exceeding 1-2 residues modified and analyzed per peptide. The other chemistries described above have not improved on this coverage.

Fourth, the use of multiple chemistries analyzed in a single experiment has never been shown. For the purposes of protein footprinting, it is not the same thing to label a protein with one chemistry and then label the protein with a second chemistry. One of the goals and impressive achievements of protein footprinting is valuable insight into solution-phase structural features. Doing sequential labeling may give some insight into the structure of the protein prior to the first labeling, but any second or subsequent labeling will not be modifying the original native protein, but rather will be modifying the already-labeled protein. The well-established idea in the field that labeling must be completed quickly for optimal results is antithetical to sequential labeling schemes intended to increase coverage. Only multiple independent experiments with different chemistries can be used to extend coverage in this way.

A need exists for a protein footprinting chemistry that achieves labeling of a greater number of amino acid side chains from the standpoint of detectible reactivity (e.g. where up to 15/20 or 18/20 of the known side chains can be probed in a single experiment), and provides high overall labeling coverage in a single-pot experiment where more than 50% of the total surface accessible residues within the individual peptides (e.g. >5/10 surface accessible residues or >8/15 surface accessible residues for example) are detected to be modified and can be quantitatively analyzed.

### SUMMARY

In an aspect, the present disclosure provides a one-pot method of multiplex protein labeling that is suitable for protein footprinting. The method includes: a) generating hydroxyl radicals in a reaction solution; and b) maintaining a hydroxyl radical concentration for a first length of time. The reaction solution includes hydroxyl radical precursors, trifluoromethyl radical precursors, and a protein of interest. The hydroxyl radicals generate trifluoromethyl radicals from the trifluoromethyl radical precursors. The first length of time is sufficient to both: (i) react a first portion of the hydroxyl radicals with the trifluoromethyl radical precursors in the reaction solution to maintain a trifluoromethyl radical concentration for a second length of time sufficient to react with the protein of interest; and (ii) react a second portion of the hydroxyl radicals with the protein of interest. Step b) thereby labels the protein of interest with both hydroxyl substituents and trifluoromethyl substituents.

In another aspect, the present disclosure provides a one-pot method of multiplex protein labeling that is suitable for protein footprinting. The method includes: a) simultaneously generating hydroxyl radicals and trifluoromethyl radicals in the presence of a protein of interest; and b) maintaining a hydroxyl radical concentration for a first length of time. The first length of time is sufficient to both: (i) react a first portion of the hydroxyl radicals with the trifluoromethyl radical precursors to maintain a trifluoromethyl radical concentration for a second length of time sufficient to react with the protein of interest; and (ii) react a second portion of the hydroxyl radicals with the protein of interest. Step b) thereby labels the protein of interest with both hydroxyl substituents and trifluoromethyl substituents.

In a further aspect, the present disclosure provides a one-pot method of multiplex protein labeling that is suitable for protein footprinting. The method includes: a) introducing a reaction solution into a radical generation chamber; b) generating hydroxyl radicals in the reaction solution; and c) maintaining a hydroxyl radical concentration in the solution for a first length of time. The reaction solution includes hydroxyl radical precursors, trifluoromethyl radical precursors, and a protein of interest. The hydroxyl radicals generated in step b) generate trifluoromethyl radicals from the trifluoromethyl radical precursors. The first length of time is sufficient to both: (i) react a first portion of the hydroxyl radicals with the trifluoromethyl radical precursors in the reaction solution to maintain a trifluoromethyl radical concentration for a second length of time sufficient to react with the protein of interest; and (ii) react a second portion of the hydroxyl radicals with the protein of interest. Step c) thereby labels the protein of interest with both hydroxyl substituents and trifluoromethyl substituents.

In yet another aspect, the present disclosure provides a one-pot method of multiplex protein labeling that is suitable for protein footprinting. The method includes: a) generating hydroxyl radicals in the presence of trifluoromethyl radical precursors and a protein of interest; and b) waiting a length of time sufficient for the hydroxyl radicals and the trifluoromethyl radicals to react with the protein of interest. The generating of step a) has a time-generation profile that is adapted to provide a combined hydroxyl and trifluoromethyl radical time-concentration profile. The waiting of step b) thereby labels the protein of interest with both hydroxyl substituents and trifluoromethyl substituents.

In another aspect, the present disclosure provides a one-pot method of multiplex protein labeling that is suitable for protein footprinting. The method includes: a) preparing a reaction sample; b) generating hydroxyl radicals in the reaction sample; and c) maintaining a hydroxyl radical concentration in the reaction sample for a length of time sufficient for the hydroxyl radicals and the trifluoromethyl radicals to react with a protein of interest. The reaction sample include hydroxyl radical precursors, trifluoromethyl radical precursors, and the protein of interest. The hydroxyl radicals generated in step b) thereby generate trifluoromethyl radicals. The maintaining of step c) thereby labels the protein of interest with both hydroxyl substituents and trifluoromethyl substituents. The relative and absolute concentrations of the hydroxyl radical precursors, the trifluoromethyl radical precursors, and the protein of interest, a volume of the sample, and the dynamics of the generating hydroxyl radicals of step b) are adapted to provide labeling of at least 16, at least 17, at least 18, at least 19, or at least 20 distinct amino acids of the 20 most abundant amino acids found in proteins of the protein of interest and at least 50% of the total surface accessible residues in the protein of interest.

In an additional aspect, the present disclosure provides a kit. The kit includes a trifluoromethyl radical precursor and instructions. The instructions are regarding reaction conditions necessary to perform a one-pot multiplex protein labeling reaction that is suitable for protein footprinting. The reaction conditions include timing information related to generation of hydroxyl radicals.

In a further aspect, the present disclosure provide a system. The system includes a reaction system, a mass spectrometer, a processor, and a memory. The reaction system includes a radical generation chamber, a hydroxyl radical generator, an inlet, an optional hydroxyl radical precursor source, an optional trifluoromethyl radical precursor source, an optional quenching agent source, and an outlet. The hydroxyl radical generation chamber is adapted to generate hydroxyl radicals from hydroxyl radical precursors in the reaction chamber. The inlet is adapted to receive a sample comprising a protein of interest and to provide the sample to the radical generation chamber. The outlet is adapted to introduce at least a portion of the contents of the radical generation chamber into the mass spectrometer or an optional pre-processing system for the mass spectrometer. The optional pre-processing system eventually introducing the at least a portion of the contents of the radical generation chamber into the mass spectrometer. The memory having stored thereon instructions that, when executed by the processor, cause the processor to: a) optionally send a radical generation signal to the hydroxyl radical generator; b) optionally send a sample transfer signal to the outlet; c) optionally send a data acquisition signal to the mass spectrometer; d) receive mass spectrometry data from the mass spectrometer; and e) identify hydroxyl-radical-modified amino acids and trifluoromethyl-radical-modified amino acids from the amino acid sequence. The instructions, when executed by the processor, can further cause the processor to: f) retrieve the amino acid sequence of the protein of interest; and g) generate a report with the hydroxyl-radical-modified amino acids and trifluoromethyl-radical-modified amino acids. Either: i) the system includes an input for identifying reaction conditions being utilized in the reaction system, the instructions, when executed by the processor, further cause the processor to utilize the reaction conditions in step e); or ii) the memory has stored thereon one or more reaction conditions for which the reaction system and/or step e) are adapted to function.

In another aspect, the present disclosure provides an auxiliary device for pairing with a mass spectrometry system. The auxiliary device includes a radical generation chamber, a hydroxyl radical generator, an inlet, an optional hydroxyl radical precursor dispenser, an optional trifluoromethyl radical precursor dispenser, an optional quenching agent dispenser, and an outlet. The hydroxyl radical generator is adapted to generate hydroxyl radicals from hydroxyl radical precursors in the radical generation chamber. The inlet is adapted to receive a sample comprising a protein of interest and to provide the sample to the radical generation container. The outlet is adapted to deliver at least a portion of the contents of the radical generation chamber to a mass spectrometry system or a pre-processing system for the mass spectrometry system.

In a still further aspect, the present disclosure provides a kit. The kit includes an auxiliary device and a non-transitory computer readable medium. The auxiliary device is substantially as described in the previous paragraph. The non-transitory computer readable medium has instructions stored thereon that, when executed by a processor, cause the processor to: a) receive mass spectrometry data associated with a product of generation of the hydroxyl radicals in the radical generation chamber; b) retrieve an amino acid sequence of a protein of interest; c) identify hydroxyl-radical-modified amino acids and trifluoromethyl-radical-modified amino acids from the amino acid sequence; and d) generate a report with the hydroxyl-radical-modified amino acids and trifluoromethyl-radical-modified amino acids. One of the following three conditions is met: i) the auxiliary device is adapted to send reaction conditions to the processor and the instructions, when executed by the processor, further cause the processor to use the reaction conditions in step d); and/or ii) the non-transitory computer readable medium has stored thereon the reaction conditions and the auxiliary device is adapted to retrieve the reaction conditions and use the reaction chambers to generate hydroxyl radicals in the radical generation chamber; and/or iii) the auxiliary device is adapted to operate with the reaction conditions, the non-transitory computer readable medium has stored thereon the reaction conditions, and the instructions, when executed by the processor, further cause the processor to use the reaction conditions in step d).

In yet another aspect, the present disclosure provides a non-transitory computer readable medium having stored thereon instructions. When the instructions are executed by a processor, they cause the processor to: a) receive mass spectrometry data; b) receive one-pot protein labeling reaction conditions; c) retrieve an amino acid sequence of a protein of interest; d) identify hydroxyl-radical-modified amino acids and trifluoromethyl-radical-modified amino acids from the amino acid sequence; and e) generate a report with the hydroxyl-radical-modified amino acids and trifluoromethyl-radical-modified amino acids.

### BRIEF DESCRIPTIONS OF THE DRAWINGS

FIG. 1 is a flowchart illustrating a method, in accordance with aspects of the present disclosure.

FIG. 2 is a flowchart illustrating a method, in accordance with aspects of the present disclosure.

FIG. 3 is a flowchart illustrating a method, in accordance with aspects of the present disclosure.

FIG. 4 is a flowchart illustrating a method, in accordance with aspects of the present disclosure.

FIG. 5 is a flowchart illustrating a method, in accordance with aspects of the present disclosure.

FIG. 6 is a diagram of a system, in accordance with aspects of the present disclosure.

FIG. 7 includes plots showing multiplex labeling on amino acid side chains, as described in Example 1.

FIG. 8 is a plot illustrating the linearity of TFM modifications on amino acids, as described in Example 1.

FIG. 9 is a pair of orthogonal chromatography plots for analysis of TFM and hydroxyl modifications, as described in Example 1.

FIG. 10 is a plot illustrating residue activity in Langlois reagent as analyzed with a C₁₈ column, as described in Example 1.

FIG. 11 is a Venn diagram illustrating the TFM and OH modifications for amino acids in Langlois reagent as analyzed with C₁₈ and HILIC columns, as described in Example 1.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Recently, the FPOP experiment described above was utilized to show trifluoromethyl radical footprinting. In this experiment the hydroxyl radicals generated by fast (nanosecond) photolysis of peroxide were allowed to react with solution based trifluoromethylation precursors, and CF₃ radicals were transferred to specifically detected protein sites. This demonstrated a fourth major footprinting chemistry (e.g. trifluoromethylation) added to the above set. This chemistry did not significantly extend coverage as a single reagent, nor did it extend coverage to increase the numbers of residues examined within individual peptides. Also, the reaction was slow (e.g. seconds) potentially compromising one of the important tenets of footprinting listed above. This slow reaction is not explained as a fast photolysis pulse was employed.

The inventors then advanced the TFM labeling by extending the experiments to radical generation via synchrotron x-ray source. In this work, proteins were labeled separately with OH radicals and TFM radicals. To a layperson, this may seem like all that is missing from the equation now is doing both OH radical and TFM radical footprinting in a single reaction, but this grossly oversimplifies the situation and fails to account for the complexity introduced by unknown reaction rates, especially those governing radical initiation and transfer.

Given that one of the labeling radicals, the OH radical, is a reactant for the formation of another labeling radical, the TFM radical, there are many reaction rates that need to be considered to have any expectations regarding the outcome. The amount (molecules/moles) of OH radical generation (in one exemplified case, from the x-ray beam) is relevant as it is a known function of the penetration depth of the sample and energy of the radiation, governing the energy deposition. The duration of OH radical generation pulse (microsecond vs. millisecond) is relevant. The amount of TFM radical generation from the OH radicals, based on chemical reactivity of radical transfer, is relevant. The type of electron donating groups bonded to the S-atom of sodium triflinate, a well-known TFM reagent, is relevant to OH reactivity and transfer. The type of atom, Si vs. S chosen as binding ligand to CF₃ in triflinate type reagents, is relevant to OH reactivity and thus CF₃ yield. The rate of reaction between OH radicals and each of 20 potential amino acid side chain targets is relevant. The rate of reaction between TFM radicals and each of the 20 potential amino acid side chain targets is relevant. Other chemical speciation and chemical features of the TFM reagent (Si vs. S design), alternative homolysis leaving groups (none, vs. Cl, vs. I), scavenging properties of scaffold, are relevant. If any specific chemistry dominates, then a single pot experiment is functionally impossible. The inventors surprisingly discovered a single pot reaction that is capable of labeling at least 13, at least 14, at least 15, or at least 16, at least 17, at least 18, at least 19, or at least 20 residues of the 20 total in proteins and at least 5, at least 6, and least 7 or at least 8, or at least 9 residues in peptides of 10 residues in length.

Two different types of coverage in labeling are contemplated here. In one case, there is coverage for a given amino acid (i.e., will a given amino acid out of the 20 be labeled). In the other case, there is overall coverage for the amino acids contained within a given protein (i.e., what overall percent of amino acids in a protein will be labeled). The standard of labeling identification includes analyzing the tandem mass spectrum using a typical search engine (Mascot, Mass Matrix) that can identify the site of labeling with a false positive rate of 1%.

The present disclosure provides a variety of methods. It should be appreciated that the methods can be combinable with one another and features of one method are expressly contemplated as being usable within the other methods, unless the context clearly dictates otherwise.

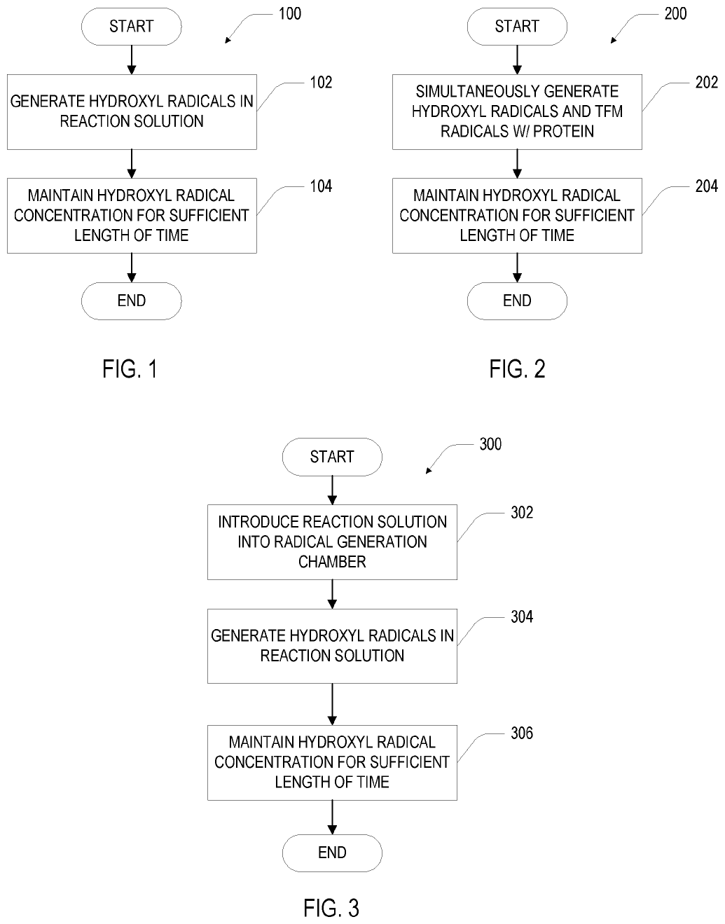

Referring to FIG. 1, a one-pot method 100 of multiplex protein labeling that is suitable for protein footprinting is disclosed. At process block 102, the method 100 includes generating hydroxyl radicals in a reaction solution. The reaction solution includes hydroxyl radical precursors, TFM precursors, and a protein of interest. The hydroxyl radical precursors are activated providing hydroxyl radicals. The hydroxyl radicals generate TFM radicals from the TFM radical precursors. At process block 104, the method 100 includes maintaining the hydroxyl radical concentration for a first length of time sufficient to both: i) react a first portion of the hydroxyl radicals with the trifluoromethyl radical precursors in the reaction solution to maintain a trifluoromethyl radical concentration for a second length of time sufficient to react with the protein of interest; and ii) react a second portion of the hydroxyl radicals with the protein of interest. This reacting thereby labels the protein of interest with both hydroxyl substituents and TFM substituents.

Referring to FIG. 2, a one-pot method 200 of multiplex protein labeling that is suitable for protein footprinting is disclosed. At process block 202, the method 200 includes simultaneously generating hydroxyl radicals and TFM radicals in the presence of a protein of interest. At process block 204, the method 200 includes maintaining a hydroxyl radical concentration for a first length of time sufficient to both: i) react a first portion of the hydroxyl radicals to maintain a trifluoromethyl radical concentration for a second length of time sufficient to react with the protein of interest; and ii) react a second portion of the hydroxyl radicals with the protein of interest. This reacting thereby labels the protein of interest with both hydroxyl substituents and TFM substituents.

Referring to FIG. 3, a one-pot method 300 of multiplex protein labeling that is suitable for protein footprinting is disclosed. At process block 302, the method 300 includes introducing a reaction solution into a radical generation chamber. The reaction solution includes hydroxyl radical precursors, TFM precursors, and a protein of interest. At process block 304, the method 300 includes generating hydroxyl radicals in the reaction solution. The hydroxyl radicals generate TFM radicals from the TFM radical precursors. At process block 306, the method 300 includes maintaining the hydroxyl radical concentration in the reaction solution for a first length of time sufficient to both: i) react a first portion of the hydroxyl radicals with the trifluoromethyl radical precursors in the reaction solution to maintain a trifluoromethyl radical concentration for a second length of time sufficient to react with the protein of interest; and ii) react a second portion of the hydroxyl radicals with the protein of interest. This reacting thereby labels the protein of interest with both hydroxyl substituents and TFM substituents.

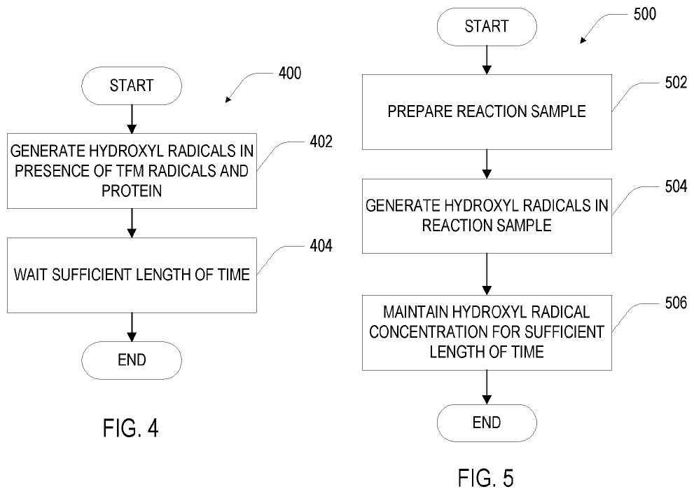

Referring to FIG. 4, a one-pot method 400 of multiplex protein labeling that is suitable for protein footprinting is disclosed. At process block 402, the method 400 includes generating hydroxyl radicals in the presence of TFM radical precursors and a protein of interest. The generating of process block 402 has a time-generation profile that is adapted to provide a combined hydroxyl and TFM radical time-concentration profile. At process block 404, the method 400 includes maintaining the hydroxyl radical concentration for a time sufficient for the hydroxyl radicals and the TFM radicals to react with the protein of interest. This reacting thereby labels the protein of interest with both hydroxyl substituents and TFM substituents. The time-generation profile and the time-concentration profile can have one or more of the properties identified below.

Referring to FIG. 5, a one-pot method 500 of multiplex protein labeling that is suitable for protein footprinting is disclosed. At process block 502, the method 500 includes preparing a reaction sample comprising hydroxyl radical precursors, TFM radical precursors, and a protein of interest. At process block 504, the method 500 includes generating hydroxyl radicals in the reaction sample. The hydroxyl radicals thereby generate TFM radicals. At process block 506, the method 500 includes maintaining the hydroxyl radical concentration for a length of time sufficient for the hydroxyl radicals and the TFM radicals to react with the protein of interest. This reacting thereby labels the protein of interest with both hydroxyl substituents and TFM substituents.

In methods 100, 200, 300, 400, 500, the following factors are adapted to provide labeling of at least 13 different residues (out of the 20 most common amino acids) of the protein of interest and to label at least 50% of the overall residues on the protein of interest: the relative and absolute concentrations of the hydroxyl radical precursors, the type and concentrations of TFM precursors, and the protein of interest; a volume of the sample; and the dynamics of the generating hydroxyl radicals of process block 504.

In some cases, the above-referenced factors of methods 100, 200, 300, 400, 500 are adapted to provide labeling of at least 13, at least 14, at least 15, or at least 16, at least 17, at least 18, at least 19, or at least 20 different residues (out of the 20 most common amino acids) of the protein of interest.

In some cases, the above-reference factors of methods 100, 200, 300, 400, 500 are adapted to provide labeling of at least 50%, at least 55%, at least 60%, at least 65%, at least 70%, at least 75%, or at least 80%, at least 85%, or at least 90% of total surface accessible residues within the protein of interest. As used herein, “surface accessible” refers to a residue that is capable of being contacted by a constituent of an aqueous solution that contains the protein of interest. A person having ordinary skill in the art will recognize that the surface accessible residues may change based on changing conditions (e.g., changing temperature may cause the protein to denature, thereby causing some previously non-accessible residues to become surface accessible, binding the protein with another molecule and/or protein may cause a conformational change in the protein of interest and cause more or less residues to be surface accessible, etc.).

The labeling of proteins of interest is achieved by balancing reaction conditions with respect to the relevant rates discussed above. This balance is described herein in a variety of ways, which are consistent with one another and should not be interpreted as describing alternatives to one another. For example, the descriptions relating to time-generation profiles and time-concentration profiles are wholly consistent with the descriptions relating to chemical flux. Without wishing to be bound by any particular theory, it is believed that the specific mechanism for creating hydroxyl radicals is not critical to the function of the methods, so long as the relevant timing and concentrations are achieved. In other words, while Applicant has exemplified methods by generating hydroxyl radicals with a high-energy x-ray beam, the methods can be adapted for use with other hydroxyl radical generation mechanisms that are capable of producing the necessary time-concentration levels.

One description of the relevant reaction conditions relates to the concept of chemical flux. Broadly, the flux of the hydroxyl radicals that are generated in the methods is balanced with the concentration and chemical properties (like scavenging or electron affinity) of TFM radical precursor to provide adequate hydroxyl radicals and TFM radicals to label the protein of interest. Without wishing to be bound by any particular theory, it is believed that the reaction kinetics described herein require a “Goldilocks” condition, where the hydroxyl radicals are generated at the proper concentrations across proper time scales to both overcome scavenging effects of sample while still transferring sufficient radical flux to TFM precursors to allow them to transfer TFM modifications efficiently to proteins. The time component is particularly important. When hydroxyl radicals are generated only on nanosecond to <10 microsecond timescales as is the case with FPOP or other ultra-fast x-ray beam methods, this is quite sufficient to label proteins with hydroxyl radicals. But, the hydroxyl radicals do not have adequate lifetime to react sufficiently with TFM precursors, which are present at millimolar or lower concentrations, to permit them to react efficiently and rapidly, in turn, with proteins to permit optimal footprinting. On the other hand, x-ray beam (or photolysis) methods that have longer duration, (e.g. from the 50 microsecond to 50 millisecond timescales), provide appropriate tuning of TFM chemistry speciation, by allowing a continuous OH generation.

When the hydroxyl radicals are generated from radiolysis of water, the chemical flux that is required to achieve the disclosed multiplex labeling can be between 10¹⁵ and 10¹⁸ hydroxyl radicals generated within a 5 μL sample using pulses of ionizing radiation having a pulse width (measured as full-width at half-maximum) of between 10 ms and 200 ms. In these cases, ˜10²¹ water molecules are present in the 5 μL volume. In some cases, these pulses of ionizing radiation include sub-pulses. For example, an underlying nanosecond pulsed source that is chopped at a lower frequency can provide the pulses (those generated by the chopping) which themselves have sub-pulses within them (the underlying nanosecond pulses). By way of comparison, conventional operational parameters that are used for single-plex labeling experiments using FPOP do not generate hydroxyl radicals in amounts and time scales that are adequate for generating TFM radicals for the one-pot methods of multiple protein labeling described herein. In a conventional single-plex labeling experiment using FPOP, with a concentration of peroxide of between 1 mM and 20 mM, the chemical flux would be between 3×10¹⁵ and 6×10¹⁶ OH radical molecules generated within a 5 μL sample using pulses of light having a pulse width (measured as full-width at half-maximum) of between 1 ns and 10 μs. Although this is sufficient OH dose to conduct state of the art OH radical footprinting, the timing of radical generation in this case is too quick to generate TFM radicals in sufficient amounts for efficient TFM labeling. This is strong evidence of the challenges associated with the present disclosure—the complexity of the various reaction rates makes it extremely challenging to predict what reactions and/or reaction conditions are likely to succeed in achieving a certain kind of labeling. See below for a discussion of how to overcome these limitations in order to use photolysis with the methods described herein.

Chemical flux is defined as the amount of a substance (moles) per unit volume (concentration) per unit time transformed in a reaction. To maximize protein footprinting the “flux” of radicals must be correctly partitioned between the OH and TFM channels to label the maximum number of residues evenly. In these experiments water (55M) or peroxide (1-20 mM) hydroxyl radical precursors are converted to hydroxyl radicals by exposure to radiolysis/plasma or high intensity light, respectively. To provide for efficient labeling even for a “single-plex” labeling the minimum hydroxyl radical concentration is 1 micromolar to be delivered over time ranges of less than a microsecond to no more than 1 ms. However, a significantly higher and a specific range of chemical flux defines the needs for efficient multi reagent chemical labeling as the in initial OH radicals must be transferred to TFM reagents. Larger radical concentrations generated in shorter times or smaller radical concentrations maintained over a longer time can satisfy the criteria for balanced generation of OH radical and radical transfer to TFM precursors. For example, a 5 mM initial concentration OH radical generated (and consumed) within 10 microseconds or 10 uM initial concentration OH radical maintained for hundreds of microseconds to milliseconds can be sufficient dependent on sample characteristics and scavenging.

Another description of the relevant reaction conditions involves describing the time evolution of the various reactants. In the chemistries described herein, the time evolution of the concentration of hydroxyl radicals will have a direct impact on the time evolution of the concentration of TFM radicals, because the hydroxyl radicals are a reactant for forming the TFM radicals.

In one case, the time-generation profile of hydroxyl radicals for the “Goldilocks” condition will be described in terms of a minimum concentration to be achieved within a defined length of time. The time-generation profile of hydroxyl radicals includes a minimum concentration of at least 1 micromolar or at least 50 micromolar hydroxyl radicals or at least 1 mM hydroxyl radicals within lengths of time of 1 millisecond to 50 milliseconds or from 20 microseconds to 1 millisecond or at least 10-20 microseconds.

The time-generation profile is distinct from the time-concentration profiles described herein in that they do not consider the subtraction of radicals due to subsequent reactions (e.g., the time-generation profile for hydroxyl radicals does not consider the reduction of their actual concentration when the hydroxyl radicals react with TFM radical precursors to generate TFM radicals nor do they reflect the competing biomolecular recombination of OH radicals that also creates radical subtraction).

The time-generation profile of hydroxyl radicals and the concentration of TFM radical precursors will result in a combined time-concentration profile of hydroxyl radicals and TFM radicals suitable for a wide range of footprinting experiments and scavenging conditions.

Generating the hydroxyl radicals in the methods described herein can be achieved by radical generation methods known to produce hydroxyl radicals in concentrations and timings described herein. In one specific example, the hydroxyl radicals are generated from an ionizing radiation source.

One example of an ionizing radiation source suitable for use in the present disclosure is an x-ray synchrotron beamline. X-ray synchrotron beamlines are quasi-CW sources of radiation, operating at high frequency pulse structure (e.g. 500 MHz). The beamline magnets accelerate the synchrotron electron beam producing radiation (e.g. light). The 17-BM XFP beamline receives synchrotron light primarily as a “pink” beam X-ray radiation from a NSLS-II 3-pole wiggler source with critical energy x-ray energy=6.8 keV. A Rh-coated mirror optic located 14 m from the 3PW source and pitched at 4.2 mrad is adjustable to a toroid to for control of beam focus and power at the sample location. After accounting for the mirror reflectivity cutoff and attenuation effects of a Be window (254 μm thick) and diamond screen (100 μm thick), 4.2×10¹⁵ photons/sec of “pink beam” over an energy range of 4.5-16 keV can delivered to the sample (67 W total power) at an NSLS-II ring current of 500 mA under normal operations.

In several kinds of hydroxyl radical footprinting (HRF) experiments at XFP the beam is defocused to provide optimal coverage of a 2.5 mm diameter 5 μL droplet in the bottom of 200 μL PCR tube. Aluminum attenuators in 7 different thicknesses are available to control total photon dose on the sample, and a Uniblitz XRS6 fast shutter defines total exposure time (minimum time=10 ms). The droplet has a 1-2 mm depth providing the path length and absorption fraction of the incident x-ray beam near 50% for the critical energy (˜7 KeV). Every 100 eV energy deposited during photon absorption provides a yield of 2.7 OH radicals.

Representative examples of routine successful experiment configurations to account for varying levels of scavenging are:
Low scavenging—protein HRF of peptides (1-3 kDa), 762 um Al attenuation (flux=2.0×10¹⁴ ph/s, 3.1 W); 10, 20, and 40 ms exposures;
Medium scavenging—protein HRF of a soluble 80 kDa protein in presence of 1 mM sugar substrate, 305 um Al attenuation (flux=7.0×10¹⁴ ph/s, 11.2 W); 10, 20, and 30 ms exposures;
High Scavenging—protein HRF of large protein complex (>500K): 25 um Al attenuation (flux=3.0×10¹⁵ ph/s, power=48 W); 50, 75, 100, 200 ms exposures; and
High Scavenging in vivo—protein HRF of unattenuated (flux=4.2×10¹⁵ ph/s, 67 W); 25, 50, 100, 200 ms exposures.

Due to the CW nature of the x-ray beam, these high incident fluxes are continuously generating OH radicals throughout the millisecond exposure times, this generation is opposed by the chemical combination of OH radicals, forming peroxide, which occurs at the diffusion limit (5×10⁹ M⁻¹ s⁻¹) and is thus quite OH radical concentration dependent. These competing reactions (generation by x-rays and destruction by solution chemistry) quickly establish a steady-state equilibrium with a constant OH radical concentration from 1 micromolar to 10 to 100 micromolar concentration depending on incident beam flux detailed above. The inexhaustible supply of water at 55M allows the production of radicals to continue un-depleted. This allows tuning the OH radical concentration times exposure time to overcome scavenging in the samples outlined above. This tuning approach is also employed to supply sufficient OH radical flux to drive sufficient transfer of radicals to trifluoromethylation precursors allowing highly efficient, fast and monotonic trifluoromethylation labeling balanced by moderate OH radical labeling.

Photolysis has been a method to generate OH radicals since the 1929 with Urey's discovery of the spectrum of hydrogen peroxide and its decomposition by light, known now to yield 2 OH molecules. Hydrogen peroxide has significant absorbance from 200-280 nm and a multitude of CW or flash lamps provide light outputs suitable for generating enough light to conduct footprinting experiments as shown by Hettich and Sharp. The emission of 266 nm laser light from Nd-YAG laser is an especially convenient “initiator” of photolysis, providing a nanosecond timescale pulse of radical initiation, called FPOP (Fast Footprinting of Proteins) by Hambly and Gross. The concentration of hydrogen peroxide is typically 10-30 mM and the photon flux sufficient to photolyze 20-40% of the OH radical precursor, thus providing a nanosecond pulse of >10 mM OH radical, which decays in microseconds due to recombination with OH also reacting with side chains of peptide or protein samples. Although this pulse is quite efficient at labeling peptides and proteins rapidly, the rapid decay of the OH radical concentration makes transfer of radicals to trifluoromethylation precursors (which requires tens of microseconds to milliseconds at the typical mM concentration of precursors) challenging and is thus is insufficient for fast and monotonic trifluoromethylation labeling as seen from Gross. Approaches to overcome this limitation suggest an approach similar to x-ray approaches, provide a high frequency light source over microsecond to millisecond illumination times using hydrogen peroxide as precursor to maintain a steady concentration of OH radicals to allow sufficient time for efficient transfer of OH radicals to trifluoromethylation precursors and thus efficient multiplex labeling using photolysis.

It should be appreciated that the specific radical generation mechanism is not relevant to the downstream aspects of the methods 100, 200, 300, 400, 500. Any radical generation mechanism that provides hydroxyl radicals on the time scales described herein is suitable for use with the present disclosure.

Methods 100, 200, 300, 400, 500 provide surprisingly impressive labeling coverage when compared to existing chemistries discussed above.

One-pot TFM-based radical reaction can provide labeling of up to 19 distinct amino acids of the 20 standard amino acids in proteins. This list includes C, W, H, F, N, Y, V, I, M, A, L, R, P, T, S, D, Q, K and G. One-pot hydroxyl radical-based reaction can provide labeling of up to 14 distinct amino acids of the 20 amino acids found in proteins. These include M, C, W, H, F, Y, P, L, I, V, K, R, E and Q residues. Two-pot reaction that combined OH-radical and Langlois reagents provides labeling of the 20 amino acids including M, C, W, H, F, Y, P, L, I, V, K, R, E, Q, T, S, A, G, N, and D.

In some cases, methods 100, 200, 300, 400, 500 provide labeling coverage of at least 50% of the total surface accessible residues in the protein of interest, including but not limited to, at least 55%, at least 60%, at least 65%, at least 70%, at least 75%, or at least 80% of the total surface accessible residues in the protein of interest.

One of the limitations of one pot hydroxyl radical footprinting alluded to above, not superseded by TFM one pot methods, is that labeling within a peptide is limited to 5-20% of the residues (1-3 out of 10-20 residues). Mostly due to dynamic range limitations, the presence of a reactive residue in a peptide may dominate the ability to detect less abundant residues, thus limiting the total numbers of residues within a peptide that can be observed in the single experiment to much less than the 14 (12) residues that, in principle, can be detected for hydroxyl radical (or TFM) labeling. This limitation can be further illustrated by reference to a specific residue, M, which is 100-1000 times more reactive than less reactive residues like I or Q to hydroxyl radicals. The experiment depends on detecting modified species typically at 1% abundance, which are vastly lower for less reactive residues and often undetectable. Even worse, the M oxidation may be non linear as it is easily over oxidized and less reliable for residue specific quantification. However, the two-pot reaction creates a Goldilocks condition for residue based coverage for two reasons. First, M is low reactive to TFM and thus peptides containing an M can be assessed by the well resolved TFM modifications alone easily and without interference. Second, the diversion of OH radicals to TFM channels reduces oxidation overall, making assessment of M and other high reactive residues more useful in residue based quantification. The benefit of making the reactivity range of the residue modifications lower allows more residues to be assessed at the same time while selectivity between TFM and OH labeling enhances the ability to asses more residues per peptide than any one pot alternative.

The hydroxyl radical precursor is water for radiolysis and plasma while it is hydrogen peroxide for photolysis-based methods. The challenges of creating a time and concentration profile to assure favorable radiolysis conditions vs photolysis are quite distinct. The concentration of the radical source water, at 55M concentration is not limiting while peroxide concentrations for photolysis based methods are typically at 20 mM and generating the time and concentration profiles depend on beam or laser fluence and repetition rate (continuous wave vs. single pulse or intermediate), energy, absorption and deposition. If electron beams or plasma sources are to be used they must be quasi-CW (e.g. 1 MHz or more) to provide efficient multiplex labeling using the approaches detailed here.

The TFM radical precursor can be Langlois reagent (sodium triflinate), Ruppert's reagent, Togui's Reagent, Umemoto's reagent, triflinate chloride, other TFM radical precursors known to those having ordinary skill in the art, and combinations thereof. It should be appreciated that these precursors are understood to those having ordinary skill in the art and some are families of precursors. In some particular instances, the TFM radical precursor is Langlois reagent. The TFM radical precursor can be present in a concentration that is chosen based on balancing with the other reaction conditions to provide the desired labeling. In some cases, the TFM radical precursor is present in a concentration of between 1 mM and 5 mM (range 1), including but not limited to, a concentration of between 5 mM and 10 mM) (range 2) or between 10 and 50 mM (range 3), where most the highly OH scavenging TFM radical precursors are used in ranges 1 and 2 only and the less scavenging precursors are used in ranges 1, 2 or 3.

The protein of interest and various precursors can be contained within a buffer solution. The buffer solution can be any buffer solution known to those having ordinary skill in the art, so long as the buffer solution does not interfere with the radical chemistries described herein. The optimal low scavenging buffers have been stated in the literature (phosphate, arsenate, 1× phosphate buffered saline, ammonium acetate etc.) and these can be used at all ranges of TFM radical precursors as stated above while more scavenging buffers or buffer components (Tris buffer, nucleotide additives, DMF co-solvent, etc.) limit the TFM precursor concentrations to range 1 or range 2 only.

Each of methods 100, 200, 300, 400, 500 can include introducing a quencher to the protein of interest and/or the solution containing the protein of interest to quench the labeling reaction. Suitable quenchers will be appreciated by those having ordinary skill in the radical chemistry arts. Examples of suitable quenchers include any chemical species reactive with OH radicals introduced at an appropriate concentration, such as individual amino acids like M (e.g. methionine amide), or other additives or buffers.

Each of methods 100, 200, 300, 400, 500 can further include analyzing the labeled protein of interest and generating a report identifying hydroxyl-radical-modified amino acids and TFM-radical-modified amino acids. The labeling can be identified in a variety of ways, including but not limited to chromatography and/or antibody based separation, chemical recognition, mass, or a combination of the above approaches. Once the labeling has been analyzed and labeled residues have been identified, a report is generated including the analysis and/or the identification of the labeled residues. The report can take any form, including but not limited to, a digital file, a visual representation shown on a display, or any other means of communicating the outcome of the labeling to a user.

The extent of labeling for modified species are measured relative to unmodified species to provide quantitative “read-out” for each labeled amino acid within a specific peptide and control likely variations in instrument performance and chromatographic conditions for the separate samples. Quantification of the extent of modification for individual amino acid is achieved by extraction of ion currents of modified and unmodified peptide species derived from the LC-MS chromatograms. The extents or rates of modification for individual species are quantified by taking into account the total unmodified and modified species and deriving a fraction modified or unmodified as the dependent variable.

The sites of modification are typically established using methods of MS/MS fragmentation such as collision-induced dissociation (CID) and electron capture dissociation (ECD) coupled to bioinformatics identification. In this workflow, MS/MS spectra derived from LC-MS/MS chromatograms are searched for peptides generated by trypsin or trypsin/Asp-N digestion of protein sequences from database with allowed variable modifications (e.g. carbamidomethylation of cysteines, oxidative and TFM modifications of all 20 amino acids) using various search engines (e.g. Mass Matrix or Mascot). Lastly, MS/MS spectra for each site of proposed modification are manually examined and verified.

The present disclosure also provides systems that are adapted for use with the methods described herein. Features of the disclosure that are described in relation to the methods are contemplated for use with the systems and vice versa, unless the context clearly dictates otherwise.

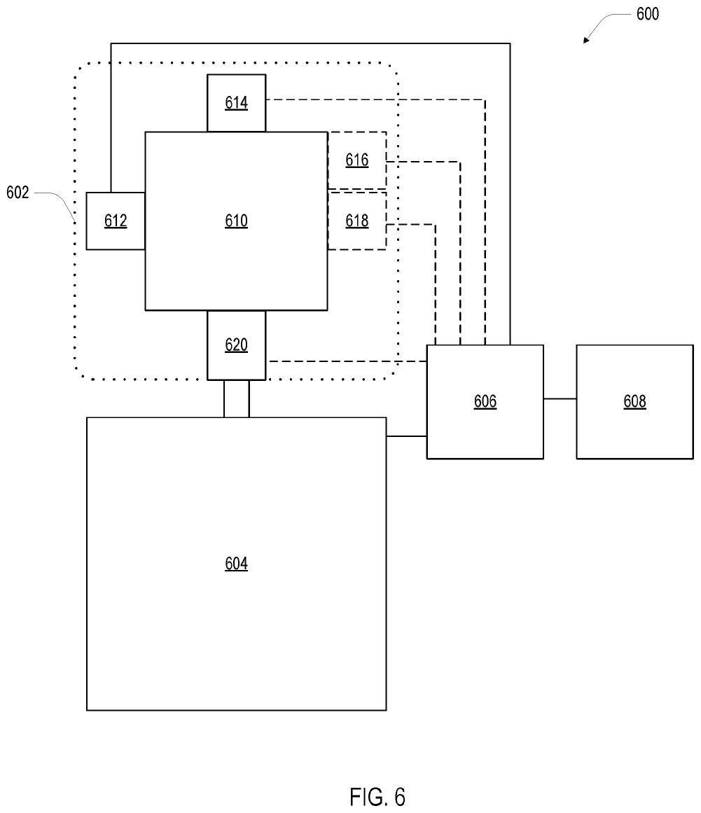

Referring to FIG. 6, a system 600 is illustrated. The system 600 includes a reaction system 602, a mass spectrometer 604, a processor 606, and a memory 608. The reaction system 602 includes a radical generation chamber 610, a hydroxyl radical generator 612, an inlet 614, an optional hydroxyl radical precursor source 616, an optional TFM radical precursor source 618, and an outlet 620.

The radical generation chamber 610 can be any container that is suitable for conducting the radical-mediated labeling described herein without unduly interfering with the chemistry. In most cases, the inner surface of the radical generation chamber 610 is chemically and biochemically inert.

The hydroxyl radical generator 612 can include an ionizing radiation source having the properties described above with respect to the methods. Other versions of the generator may incorporate devices (grounded or electrically isolated) to electrochemically generate radicals as part of the chamber's structure or include optical windows for the introduction of light using lasers or other high intensity light sources. In some cases, the hydroxyl radical generator 612 can be integrated with the radical generation chamber. In some cases, the hydroxyl radical generator 612 is separate from the radical generation chamber (e.g., when a physically large ionizing radiation source is used), in which case the reaction system 602 can include an ionizing radiation window through which ionizing radiation from the hydroxyl radical generator 612 is introduced into the radical generation chamber 610. In cases where an optical source is the hydroxyl radical generator 612 and it is separate from the radical generation chamber 610, the radical generation chamber 610 can include an optical window and any other necessary optics for directing light into the radical generation chamber 610. A person having ordinary skill in the art of radical chemistry would appreciate that multiple different radical generation mechanisms are capable of generating hydroxyl radicals on the time and concentration scales described herein and the description of system 600 is not intended to be limited to just one particular hydroxyl radical generator 612.

The inlet 614 and outlet 620 of the radical generation chamber 610 can be adapted for manual, semi-automatic, or fully automatic introduction of reactants to the chamber 610 and removal of reacted products from the chamber 610. The inlet 614 is adapted to receive at least the protein of interest, though other components of the reaction mixture can also be introduced into the chamber 610 via the inlet 614. The outlet 620 can be directly coupled to the mass spectrometer 604 to introduce the contents of the chamber 610 into the mass spectrometer 604 or the outlet 620 can be coupled to a pre-processing system that processes, separates, and/or loads samples into the mass spectrometer 604.

The optional hydroxyl radical precursor source 616 and the optional TFM radical precursor source 618, when present, are intended to allow for enhanced user experience by allowing the user to simply introduce a sample including a protein of interest into the radical generation chamber 610 without the need to mix the sample with the precursors before introduction. The sources 616, 618 can be manually operated or can be automated to dispense a chosen amount of precursor upon receipt of a signal from the processor 606.

The reaction system 602 and/or the system 600 includes the appropriate electronic, mechanical, and optical structures to provide control of the radical generation timing. In the case of ionizing radiation or optical radiation, as a non-limiting example, such a control could involve an optical shutter. In the case of devices that electrochemically generate radicals, the appropriate circuitry can be included to provide time control of the radical generation.

The reaction system 602 and/or the system 600 can optionally include a spectrometer to optically interrogate the contents of the radical generation chamber 610. Such a spectrometer can be utilized to monitor reaction progress.

The reaction system 602 and/or the system 600 can optionally include a dosimeter to monitor radical generation within the radical generation chamber 610.

The reaction system 602 and/or the system 600 can optionally include quencher reservoirs and/or sources and/or injectors (not illustrated), which are adapted to introduce a chemical quencher into the radical generation chamber to terminate the labeling reactions. The quencher reservoirs and/or sources and/or injectors can be manually operated or can be automated to dispense a chosen amount of quencher upon receipt of a signal from the processor 606.

The reaction system 602 can also be provided as a standalone auxiliary device for pairing with a commercial mass spectrometry system.

The mass spectrometer 604 can be any conventional mass spectrometer capable of distinguishing the differences in molecular weights of fragments that are formed from labeled proteins. The mass spectrometer 604 is a high-resolution mass spectrometer. Examples of suitable commercial mass spectrometers include, but are not limited to ThermoFisher Scientific instruments such as Orbitrap Eclipse, Orbitrap Fusion Lumos, Q Exactive Plus, Q Exactive HF and Orbitrap Elite, and Bruker timsTOF Pro mass spectrometer, and Waters SynaptG2.

The processor 606 can include any processing unit capable of executing the necessary commands described herein, including but not limited to, central processing units and the like. The processor 606 can be located in a computer, a smartphone, a tablet, or other similar computing devices. The processor 606 can be a single processing unit or a plurality of processing units configured to function in coordination with one another to execute the function of the processor 606.

The memory 608 can have stored thereon instructions that, when executed by the processor 606, cause the processor 606 to execute the computational aspects of the methods described herein.

In some cases, the instructions, when executed by the processor 606, cause the processor to: a) optionally send a radical generation signal to the hydroxyl radical generator 612; b) optionally send a sample transfer signal to the outlet 620; c) optionally send a data acquisition signal to the mass spectrometer 604; d) receive mass spectrometry data from the mass spectrometer 604; e) retrieve an amino acid sequence of the protein of interest; f) identify hydroxyl-radical-modified amino acids and trifluoromethyl-radical-modified amino acids from the amino acid sequence; and g) generate a report with the hydroxyl-radical-modified amino acids and trifluoromethyl-radical-modified amino acids. In some cases, the system 600 can optionally include an input for identifying reaction conditions to be utilized in the reaction system, the instructions, when executed by the processor 606, further cause the processor 606 to utilize the reaction conditions in step f). In some cases, the memory 608 has stored thereon one or more reaction conditions for which the reaction system and/or step f) are adapted to function.

In some cases, the instructions can cause the processor to optionally send a quench signal to the quencher reservoir and/or source and/or injector to trigger the introduction of quencher into the reaction chamber 610.

The present disclosure also provides kits that are suitable for use in the methods described herein and with the systems described herein. Features of the disclosure that are described in relation to the methods and systems are contemplated for use with the kits and vice versa, unless the context clearly dictates otherwise.

One version of a kit includes a TFM radical precursor and instructions. The instructions include reaction conditions necessary to perform a one-pot multiplex protein labeling reaction that is suitable for protein footprinting. The reaction conditions can include timing information related to the generation of hydroxyl radicals. This timing information can include the chemical flux and/or time-concentration profile and/or time-generation profile discussed above. In some cases, the reaction conditions can include relative proportions of hydroxyl radical precursor to TFM radical precursor. In some cases, the reaction conditions include x-ray beam parameters that are suitable for generation of the hydroxyl radicals consistent with the timing information.

Another version of a kit includes a reaction mixture and instructions. The reaction mixture includes a TFM radical precursor and a hydroxyl radical precursor. The instructions identify an amount of protein of interest to be added to and/or solubilized by the reaction mixture. The instructions include hydroxyl radical generation parameters adapted for performing a one-pot multiple protein labeling reaction that is suitable for protein footprinting.

Yet another version of a kit includes the reaction system 602 as a standalone auxiliary device and the non-transitory computer readable medium described above. The auxiliary device can be adapted to send reaction conditions to the processor and the instructions, when executed by the processor, further cause the processor to use the reaction conditions in generating a report. The non-transitory computer readable medium can have stored thereon the reaction conditions and the auxiliary device can be adapted to retrieve the reaction conditions and use the reaction conditions to generate hydroxyl radicals in the radical generation chamber. The auxiliary device can be adapted to operate with the reaction conditions, the non-transitory computer readable medium can have stored thereon the reaction conditions, and the instructions, when executed by the processor, can further cause the processor to use the reaction conditions in generating a report.

The present disclosure also provides non-transitory computer-readable media that are suitable for use in the methods, systems, and kits described herein. Features of the disclosure that are described in relation to the methods, systems, and kits are contemplated for use with the computer-readable media and vice versa, unless the context clearly dictates otherwise.

In one specific example, the non-transitory computer-readable medium has stored thereon instructions that, when executed by a processor, cause the processor to: a) receive mass spectrometry data; b) receive one-pot protein labeling reaction conditions; c) retrieve an amino acid sequence of a protein of interest; d) identify hydroxyl-radical-modified amino acids and trifluoromethyl-radical-modified amino acids from the amino acid sequence; and e) generate a report with the hydroxyl-radical-modified amino acids and trifluoromethyl-radical-modified amino acids.

### Example 1

To benchmark the chemistry of hydroxyl radical induced TFM labeling for all 20 amino acids, the inventors tested different solvent conditions (water, lx phosphate buffered saline and 2 mM ammonium acetate solution), various X-ray flux conditions of the BM-17 beamline at NSLS-II, Brookhaven National Laboratory, Upton NY (203 μm/low flux and 76 μm/high flux of aluminum attenuation), various sodium triflinate concentrations (7.5 mM and 30 mM) and various amino acid's concentrations (25 and 100 μM of AA) by measuring modification rate constants for CF3 and OH labeling. Inventors found that both 1× phosphate buffered saline, and 2 mM ammonium acetate buffers showed similar labeling in Alexa488 assay. To establish the reactivity to Langlois reagent for all 20 AAs, investors utilize 25 μM concentration of each AA in 2 mM ammonium acetate, 7.5 mM of sodium triflinate (Langlois reagent) as a TFM reagent and 76 μm of Al attenuation to produce X-ray flux on BM-17).

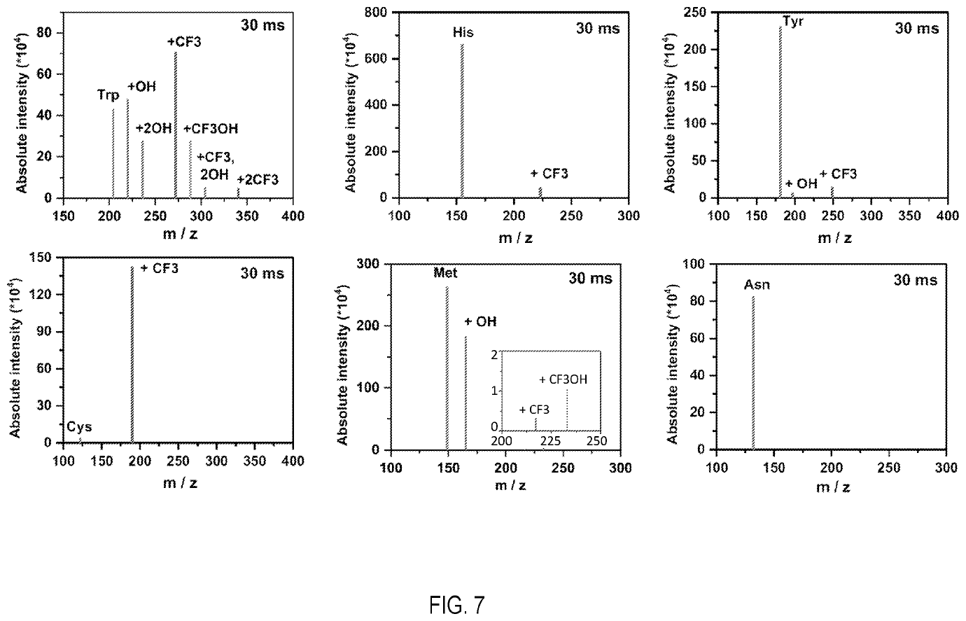

The inventors have benchmarked the chemistry of hydroxyl radical induced TFM labeling for all 20 natural amino acids by using sodium triflinate (Langlois reagent) as a TFM reagent and synchrotron X-rays for radiolysis. FIG. 7 shows the labeling intensity for several amino acids at 7.5, 15 and 30 mM sodium trfilinate reagent.

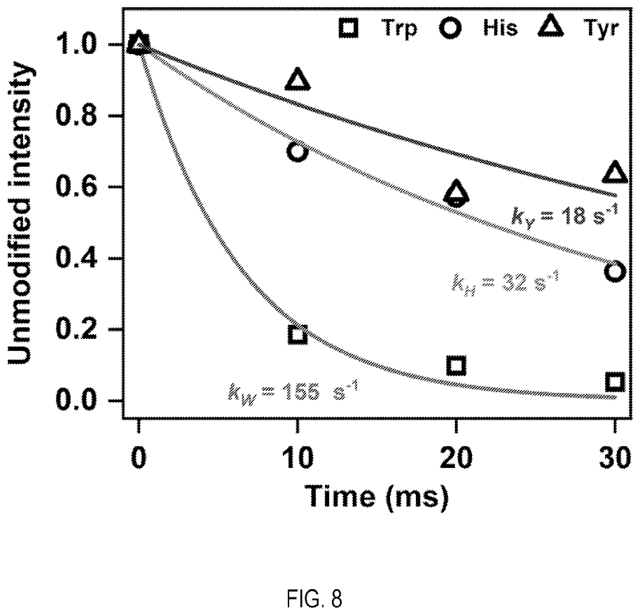

Using direct infusion mass spectrometry with a Thermo-Fisher Q-exactive or similar instrument, we found amino acids (Trp, His and Tyr) in Langlois reagent exhibited increased tri-fluoromethylation as a function of increased X-ray exposure, as illustrated in FIG. 8. Further, the modification rate constants for these amino acids were increased with reduction in the concentrations of the amino acid concentration or with increases of X-ray flux conditions, providing an evidence that TFM chemistry can be tuned similar to hydroxyl radical footprinting. Attempts to provide evidence for adducts for other amino acids than the above were less successful using infusion methods, generally due to lowered sensitivity of infusion methods for mixtures in general and the concentrations of salts.

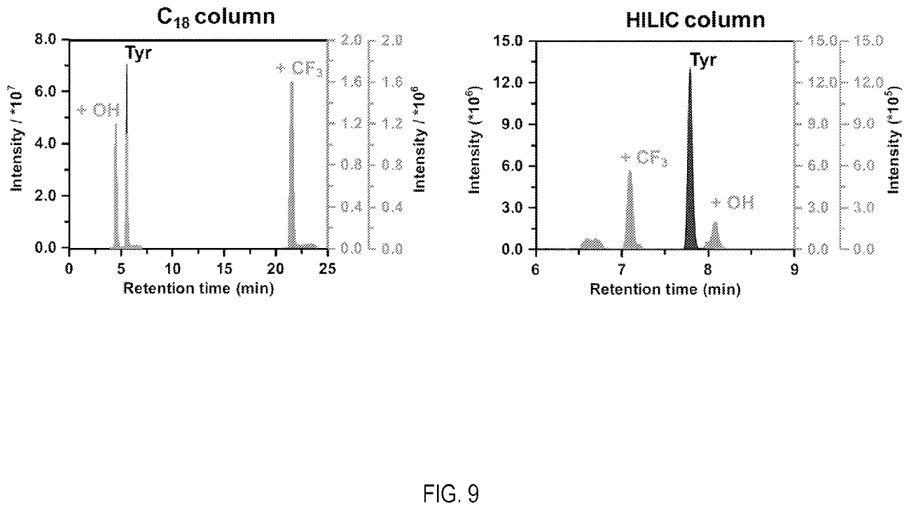

To enhance sensitivity of detection of modifications by resolving radiolysis products, we utilized established LC-chromatography methods using Thermo Fisher Q-Exactive mass spectrometry or similar instruments, that covers a wide range of polarity, and as modification product polarity cannot be reliably predicted. We found CF₃ and OH modified amino acids elute separately on polar HILIC and nonpolar C₁₈ chromatographic columns (and separately from unmodified amino acids) due to the opposite polarity of those modifications. Thus, detected modified species not only prove possibility of modification they specify by retention time and method the specific information for identifying the modifications' contribution to the retention time in general. FIG. 9 includes the data.

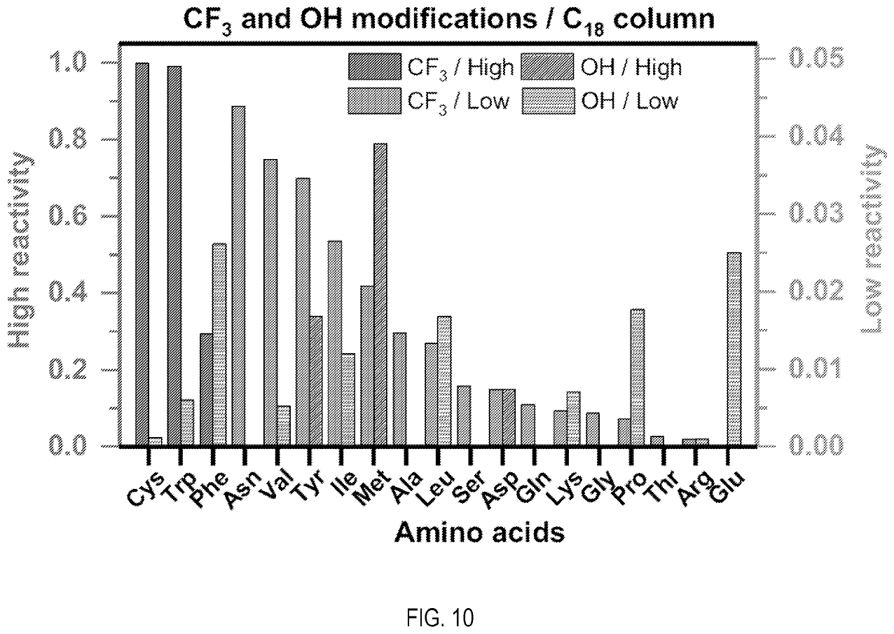

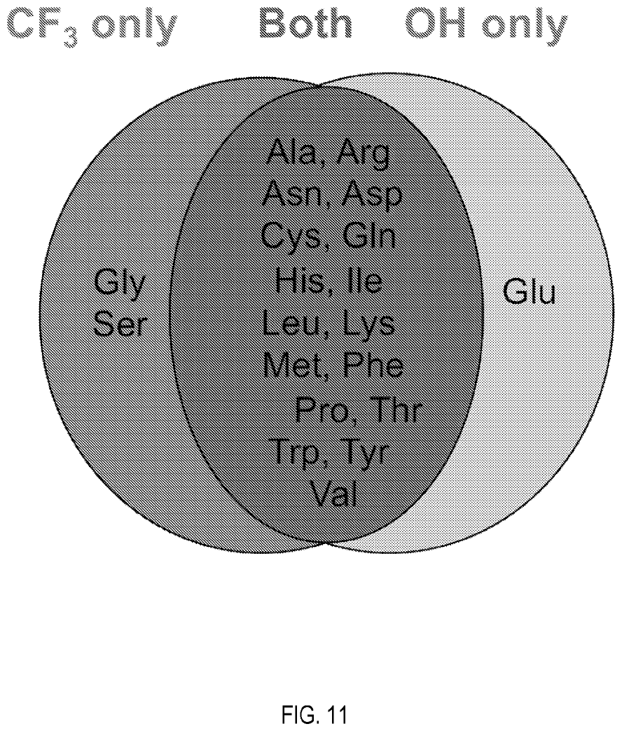

We found all 20 amino acids exhibit either CF₃ or OH labeling in presence of Langlois reagent. Those most reactive to CF₃ were cysteine, tryptophan, phenylalanine, and histidine. Amino acids with low OH reactivity in Langlois reagent (alanine, asparagine, glutamine, glycine, serine and threonine) show CF₃ reactivity. Out of 20 amino acids, 19 show CF₃ reactivity and 16 show both CF₃ and OH reactivity. Glutamic acid was the only amino acid not found to show CF₃ modifications, but it exhibited OH reactivity in Langlois reagent. FIG. 10 is a plot illustrating the reactivity of the listed amino acids (note: histidine is not shown). FIG. 11 is a Venn diagram illustrating the labeling coverage.

The high reactivity of Methionine residue for OH radicals predominantly modifies Met-residue in Met-containing sequences and silences the ability to detect any modification of other reduces. We observed Methionine amino acid in Langlois reagent shows very low CF₃ modified fraction (2%) in comparison to OH modified fraction (79%).

### Example 2

During benchmarking of sodium triflinate in ongoing studies, we had observed a significant reduction of rate constant of Alexa488 fluorescence decay in TFM labeling due to the routing of OH radicals for generating CF₃ radicals. We used this assay to test the feasibility of new TFM reagents for their labeling efficiency at different concentrations of TFM reagents and varying X-ray flux.

Out of seven new TFM reagents (Table A), three compounds (B-D) have shown a large decrease in Alexa488 decay rates at high X-ray flux conditions as compared to sodium triflinate (76 μm aluminum thickness).

Two TFM reagents (E-F) show a small change while remaining two TFM reagents (G-H) show no change in their Alexa488 decay rates in comparison to sodium triflinate.

This data provides a method for rapid screening of sodium triflinate reagents fly and efficiency of hydroxyl radicals or suitability as footprinting reagents. Sodium triflinate has high scavenging ratio (˜40 fold reduced compared to 30 mM sodium phosphate buffer or similar non-scavenging buffer) because it reacts rapidly and efficiently with hydroxyl radical and thus can “channel” substantial chemical flux towards trimethylation reactions. Reagents with low scavenging ratios cannot sufficiently re-channel hydroxy radicals to provide significantly reagent coverage.

TABLE A

| S. No. | Reagent | k_{Buffer}/k_{TFM reagent} |
|---|---|---|
| A | Sodium triflinate (Langlois reagent) | ~40 |
| B | Umemato-tetrafluoroborate | ~27 |
| C | Umemato-trifluoromethanesulfonate | ~43 |
| D | Zinc trifluoromethanesulfonate | ~49 |
| E | Ethyl trifluoromethanesulfonate | ~4 |
| F | 4,4,4,4′,4′,4′-Hexafluoro-DL-valine | ~2 |
| G | Trifluoromethanesulfonyl chloride | ~1 |
| H | Trimethyl(Trifluoromethyl)silane | ~1 |

The present disclosure has described one or more preferred embodiments, and it should be appreciated that many equivalents, alternatives, variations, and modifications, aside from those expressly stated, are possible and within the scope of the invention.

We claim:

1. A one-pot method of multiplex protein labeling that is suitable for protein footprinting, the method comprising:
a) generating hydroxyl radicals in a reaction solution, the reaction solution comprising hydroxyl radical precursors, trifluoromethyl radical precursors, and a protein of interest, the hydroxyl radicals generating trifluoromethyl radicals from the trifluoromethyl radical precursors;
b) maintaining a hydroxyl radical concentration for a first length of time sufficient to both:
i) react a first portion of the hydroxyl radicals with the trifluoromethyl radical precursors in the reaction solution to maintain a trifluoromethyl radical concentration for a second length of time sufficient to react with the protein of interest; and
ii) react a second portion of the hydroxyl radicals with the protein of interest,
thereby labeling the protein of interest with both hydroxyl substituents and trifluoromethyl substituents.

2. A one-pot method of multiplex protein labeling that is suitable for protein footprinting, the method comprising:
a) simultaneously generating hydroxyl radicals and trifluoromethyl radicals in the presence of a protein of interest.

3. The method of the immediately preceding claim, the method further comprising:
b) maintaining a hydroxyl radical concentration for a first length of time sufficient to both:
i) react a first portion of the hydroxyl radicals to maintain a trifluoromethyl radical concentration for a second length of time sufficient to react with the protein of interest; and
ii) react a second portion of the hydroxyl radicals with the protein of interest,
thereby labeling the protein of interest with both hydroxyl substituents and trifluoromethyl substituents.

4. A one-pot method of multiplex protein labeling that is suitable for protein footprinting, the method comprising:
a) introducing a reaction solution into a radical generation chamber, the reaction solution comprising hydroxyl radical precursors, trifluoromethyl radical precursors, and a protein of interest;
b) generating hydroxyl radicals in the reaction solution, the hydroxyl radicals generating trifluoromethyl radicals from the trifluoromethyl radical precursors; and
c) maintaining a hydroxyl radical concentration in the reaction solution for a first length of time sufficient to both:
i) react a first portion of the hydroxyl radicals with the trifluoromethyl radical precursors in the reaction solution to maintain a trifluoromethyl radical concentration for a second length of time sufficient to react with the protein of interest; and
ii) react a second portion of the hydroxyl radicals with the protein of interest,
thereby labeling the protein of interest with both hydroxyl substituents and trifluoromethyl substituents.

5. A one-pot method of multiplex protein labeling that is suitable for protein footprinting, the method comprising:
a) generating hydroxyl radicals in the presence of trifluoromethyl radical precursors and a protein of interest, the generating having a time-generation profile that is adapted to provide a combined hydroxyl and trifluoromethyl radical time-concentration profile; and
b) waiting a length of time sufficient for the hydroxyl radicals and the trifluoromethyl radicals to react with the protein of interest, thereby labeling the protein of interest with both hydroxyl substituents and trifluoromethyl substituents.

6. The method of claim 5, wherein the time-generation profile includes a hydroxyl radical concentration of at least 1 μM for a length of time from 1 ms to 50 ms.

7. The method of claim 5 or 6, wherein the time-generation profile includes a hydroxyl radical concentration of at least 50 μM within a length of time of at least 20 s and as long as 1 ms.

8. The method of any one of claim 5 to the immediately preceding claim, wherein the time-generation profile includes a hydroxyl radical concentration of at least 1 mM within a length of time of at least 20 μs.

9. The method of any one of claim 5 to the immediately preceding claim, wherein the time-generation profile includes a hydroxyl radical concentration of between 1 μM and 5 mM within a time range of between 20 μs and 50 ms.

10. A one-pot method of multiplex protein labeling that is suitable for protein footprinting, the method comprising:
a) preparing a reaction sample comprising hydroxyl radical precursors, trifluoromethyl radical precursors, and a protein of interest;
b) generating hydroxyl radicals in the reaction sample, the hydroxyl radicals thereby generating trifluoromethyl radicals; and
c) maintaining the hydroxyl radical concentration for a length of time sufficient for the hydroxyl radicals and the trifluoromethyl radicals to react with the protein of interest, thereby labeling the protein of interest with both hydroxyl substituents and trifluoromethyl substituents, wherein the relative and absolute concentrations of the hydroxyl radical precursors, the trifluoromethyl radical precursors, and the protein of interest, a volume of the sample, and the dynamics of the generating hydroxyl radicals of step b) are adapted to provide labeling of at least 16, at least 17, at least 18, at least 19, or at least 20 distinct amino acids of the 20 most abundant amino acids found in proteins of the protein of interest and at least 50% of the total surface accessible residues in the protein of interest.

11. The method of any one of the preceding claims, the method further comprising: adding a quenching agent to the reaction solution and/or the presence of the protein of interest and/or the reaction sample, thereby quenching the multiplex protein labeling.

12. The method of any one of the preceding claims, wherein generating hydroxyl radicals comprises irradiating the hydroxyl radical precursor or the hydroxyl radical precursors with ionizing radiation.

13. The method of any one of claims 1 to 11, wherein generating hydroxyl radicals comprises irradiating the hydroxyl radical precursor or the hydroxyl radical precursors with photolytic radiation.

14. The method of any one of claims 1 to 11, wherein generating hydroxyl radicals comprises electrochemically generating hydroxyl radicals.

15. A kit comprising:
a reaction mixture comprising a trifluoromethyl radical precursor and an optional hydroxyl radical precursor; and
instructions identifying an amount of a protein of interest to be added to and/or solubilized by the reaction mixture, the instructions including hydroxyl radical generation parameters adapted for performing a one-pot multiple protein labeling reaction that is suitable for protein footprinting.

16. A kit comprising:
a trifluoromethyl radical precursor; and
instructions regarding reaction conditions necessary to perform a one-pot multiplex protein labeling reaction that is suitable for protein footprinting, the reaction conditions including timing information related to generation of hydroxyl radicals.

17. The kit of the immediately preceding claim, the reaction conditions including relative proportions of hydroxyl radical precursor to trifluoromethyl radical precursor.

18. The kit of the immediately preceding claims, the reaction conditions including ionizing radiation parameters that are suitable for generation of the hydroxyl radicals consistent with the timing information.

19. The kit of claim 17, the reaction conditions including electrochemical parameters that are suitable for generation of the hydroxyl radicals consistent with the timing information.

20. The kit of claim 17, the reaction conditions including optical parameters that are suitable for generation of the hydroxyl radicals consistent with the timing information.

21. A system comprising a reaction system, a mass spectrometer, a processor, and a memory, the reaction system comprising:
a radical generation chamber to generate hydroxyl radicals and trifluoromethyl radicals simultaneously;
a hydroxyl radical generator adapted to generate the hydroxyl radicals from hydroxyl racial precursors in the radical generation chamber;
an inlet adapted to receive a sample comprising a protein of interest and to provide the sample to the radical generation chamber;
an optional hydroxyl radical precursor source;
an optional trifluoromethyl radical precursor source;
an optional quenching agent source; and
an outlet adapted to introduce at least a portion of the contents of the radical generation chamber into the mass spectrometer or an optional pre-processing system for the mass spectrometer, the optional pre-processing system eventually introducing the at least a portion of the contents of the radical generation chamber into the mass spectrometer,
the memory having stored thereon instructions that, when executed by the processor, cause the processor to:
a) optionally send a radical generation signal to the hydroxyl radical generator;
b) optionally send a sample transfer signal to the outlet;
c) optionally send a data acquisition signal to the mass spectrometer;
d) receive mass spectrometry data from the mass spectrometer;
e) identify hydroxyl-radical-modified amino acids and trifluoromethyl-radical-modified amino acids from an amino acid sequence of the protein of interest.

22. The method of the immediately preceding claim, the method further comprising:
f) retrieve the amino acid sequence of the protein of interest; and
g) generate a report with the hydroxyl-radical-modified amino acids and trifluoromethyl-radical-modified amino acids,
wherein either:
i) the system includes an input for identifying reaction conditions being utilized in the reaction system, the instructions, when executed by the processor, further cause the processor to utilize the reaction conditions in step e); or
ii) the memory has stored thereon one or more reaction conditions for which the reaction system and/or step e) are adapted to function.

23. An auxiliary device for pairing with a mass spectrometry system, the auxiliary device comprising:
a radical generation chamber;
a hydroxyl radical generator adapted to generate hydroxyl radicals from hydroxyl radical precursors in the radical generation chamber;
an inlet adapted to receive a sample comprising a protein of interest and to provide the sample to the radical generation container;
an optional hydroxyl radical precursor dispenser;
an optional trifluoromethyl radical precursor dispenser;
an optional quenching agent dispenser; and
an outlet adapted to deliver at least a portion of the contents of the radical generation chamber to a mass spectrometry system or a pre-processing system for the mass spectrometry system.

24. A kit comprising an auxiliary device and a non-transitory computer readable medium, the auxiliary device comprising:
a radical generation chamber;
a hydroxyl radical generator adapted to generate hydroxyl radicals from hydroxyl radical precursors in the radical generation chamber;
an inlet adapted to receive a sample and to provide the sample to the radical generation chamber; and
an outlet adapted to deliver at least a portion of the contents of the radical generation chamber to a mass spectrometry system or a pre-processing system for the mass spectrometry system,
the non-transitory computer readable medium having stored thereon instructions that, when executed by a processor, cause the processor to:
a) receive mass spectrometry data associated with a product of generation of the hydroxyl radicals in the radical generation chamber;
b) retrieve an amino acid sequence of a protein of interest;
c) identify hydroxyl-radical-modified amino acids and trifluoromethyl-radical-modified amino acids from the amino acid sequence; and
d) generate a report with the hydroxyl-radical-modified amino acids and trifluoromethyl-radical-modified amino acids,
wherein:
i) the auxiliary device is adapted to send reaction conditions to the processor and the instructions, when executed by the processor, further cause the processor to use the reaction conditions in step d); and/or
ii) the non-transitory computer readable medium has stored thereon the reaction conditions and the auxiliary device is adapted to retrieve the reaction conditions and use the reaction chambers to generate hydroxyl radicals in the radical generation chamber; and/or
iii) the auxiliary device is adapted to operate with the reaction conditions, the non-transitory computer readable medium has stored thereon the reaction conditions, and the instructions, when executed by the processor, further cause the processor to use the reaction conditions in step d).

25. The system, auxiliary device, or kit of any one of claim 21 to the immediately preceding claim, further comprising a dosimeter adapted to monitor radical generation within the radical generation chamber.

26. The system, auxiliary device, or kit of any one of claim 21 to the immediately preceding claim, further comprising spectrometer adapted to optically interrogate the contents of the radical generation chamber.

27. The system, auxiliary device, or kit of the immediately preceding claim, wherein the spectrometer provides feedback to the processor.

28. The system, auxiliary device, or kit of the immediately preceding claim, wherein the processor controls the hydroxyl radical generator utilizing the feedback.

29. The system, auxiliary device, or kit of any one of claim 21 to the immediately preceding claim, comprising the hydroxyl radical precursor source or dispenser.

30. The system, auxiliary device, or kit of the immediately preceding claim, wherein the hydroxyl radical precursor source or dispenser is automated to dispense a chosen amount of hydroxyl radical precursor upon receipt of a signal.

31. The system, auxiliary device, or kit of any one of claim 21 to the immediately preceding claim, comprising the trifluoromethyl radical precursor source or dispenser.

32. The system, auxiliary device, or kit of the immediately preceding claim, wherein the trifluoromethyl radical precursor source or dispenser is automated to dispense a chosen amount of hydroxyl radical precursor upon receipt of a signal.

33. The system, auxiliary device, or kit of any one of claim 21 to the immediately preceding claim, comprising the quenching agent source or dispenser.

34. The system, auxiliary device, or kit of the immediately preceding claim, wherein the quenching agent source or dispenser is automated to dispense a chosen amount of hydroxyl radical precursor upon receipt of a signal.

35. The system, auxiliary device, or kit of any one of claim 21 to the immediately preceding claim, comprising a radical generation source.

36. The system, auxiliary device, or kit of claim 35, wherein the radical generation source is an ionizing radiation source, the radical generation chamber comprising an ionizing radiation window through which the ionizing radiation source introduces ionizing radiation into the radical generation chamber.

37. The system, auxiliary device, or kit of claim 35, wherein the radical generation source is a photolytic radiation source, the radical generation chamber comprising a photolytic radiation window through which the photolytic radiation source introduces photolytic radiation into the radical generation chamber.

38. The system, auxiliary device, or kit of claim 35, wherein the radical generation source is an electrochemical system positioned within the radical generation chamber or adapted to introduce an electrochemical radical generation mechanism into the radical generation chamber.

39. A non-transitory computer readable medium having stored thereon instructions that, when executed by a processor, cause the processor to:
a) receive mass spectrometry data;
b) receive one-pot protein labeling reaction conditions;
c) retrieve an amino acid sequence of a protein of interest;
d) identify hydroxyl-radical-modified amino acids and trifluoromethyl-radical-modified amino acids from the amino acid sequence; and
e) generate a report with the hydroxyl-radical-modified amino acids and trifluoromethyl-radical-modified amino acids.

40. The method, system, auxiliary device, kit, or non-transitory computer readable medium of any one of the preceding claims, wherein labeling the protein of interest comprises labeling at least 13 distinct amino acids of the 20 most abundant amino acids found in proteins.

41. The method, system, auxiliary device, kit, or non-transitory computer readable medium of any one of the preceding claims, wherein labeling the protein of interest comprises labeling at least 14 distinct amino acids of the 20 most abundant amino acids found in proteins.

42. The method, system, auxiliary device, kit, or non-transitory computer readable medium of any one of the preceding claims, wherein labeling the protein of interest comprises labeling at least 15 distinct amino acids of the 20 most abundant amino acids found in proteins.

43. The method, system, auxiliary device, kit, or non-transitory computer readable medium of any one of the preceding claims, wherein labeling the protein of interest comprises labeling at least 16 distinct amino acids of the 20 most abundant amino acids found in proteins.

44. The method, system, auxiliary device, kit, or non-transitory computer readable medium of any one of the preceding claims, wherein labeling the protein of interest comprises labeling at least 17 distinct amino acids of the 20 most abundant amino acids found in proteins.

45. The method, system, auxiliary device, kit, or non-transitory computer readable medium of any one of the preceding claims, wherein labeling the protein of interest comprises labeling at least 18 distinct amino acids of the 20 most abundant amino acids found in proteins.

46. The method, system, auxiliary device, kit, or non-transitory computer readable medium of any one of the preceding claims, wherein labeling the protein of interest comprises labeling at least 19 distinct amino acids of the 20 most abundant amino acids found in proteins.

47. The method, system, auxiliary device, kit, or non-transitory computer readable medium of any one of the preceding claims, wherein labeling the protein of interest comprises labeling at least 20 distinct amino acids of the 20 most abundant amino acids found in proteins.

48. The method, system, auxiliary device, kit, or non-transitory computer readable medium of any one of the preceding claims, wherein labeling the protein of interest comprises labeling at least 50% of total surface accessible residues within the protein of interest.

49. The method, system, auxiliary device, kit, or non-transitory computer readable medium of any one of the preceding claims, wherein labeling the protein of interest comprises labeling at least 60% of total surface accessible residues within the protein of interest.

50. The method, system, auxiliary device, kit, or non-transitory computer readable medium of any one of the preceding claims, wherein labeling the protein of interest comprises labeling at least 70% of total surface accessible residues within the protein of interest.

51. The method, system, auxiliary device, kit, or non-transitory computer readable medium of any one of the preceding claims, wherein labeling the protein of interest comprises labeling at least 80% of total surface accessible residues within the protein of interest.

52. The method, system, auxiliary device, kit, or non-transitory computer readable medium of any one of the preceding claims, wherein labeling the protein of interest comprises labeling at least 90% of total surface accessible residues within the protein of interest.

53. The method, system, auxiliary device, kit, or non-transitory computer readable medium of any one of the preceding claims, wherein labeling the protein of interest comprises a time-generation profile of hydroxyl radicals including a concentration of at least 1 μM, at least 50 μM, or at least 5 mM within a length of time of less than 1 ms, less than 500 μs, or less than 1 μs.

54. The method, system, auxiliary device, kit, or non-transitory computer readable medium of any one of the preceding claims, wherein labeling the protein of interest comprises a time-generation profile of hydroxyl radicals including a concentration of between 1 μM and 5 mM within a time range of between 10 μs to 50 ms.

55. The method, system, auxiliary device, kit, or non-transitory computer readable medium of any one of the preceding claims, wherein the hydroxyl radical precursor comprises water or hydrogen peroxide or the hydroxyl radical precursors comprise water or hydrogen peroxide.

56. The method, system, auxiliary device, kit, or non-transitory computer readable medium of any one of the preceding claims, wherein the hydroxyl radical precursor comprises water or the hydroxyl radical precursors comprise water.

57. The method, system, auxiliary device, kit, or non-transitory computer readable medium of any one of the preceding claims, wherein the hydroxyl radical precursor or the hydroxyl radical precursors are present in excess quantity.

58. The method, system, auxiliary device, kit, or non-transitory computer readable medium of any one of the preceding claims, wherein the trifluoromethyl radical precursor comprises or the trifluoromethyl radical precursors comprise a precursor selected from the group consisting of Langlois reagent, umemato-tetrafluoroborate, umemato-trifluoromethanesulfonate, zinc trifluoromethanesulfonate, ethyl trifluoromethanesulfonate, 4,4,4,4′,4′,4′-hexafluoro-DL-valine, or a combination thereof.

59. The method, system, auxiliary device, kit, or non-transitory computer readable medium of any one of the preceding claims, wherein the trifluoromethyl radical precursor comprises Langlois reagent or the trifluoromethyl radical precursors comprise Langlois reagent.

60. A method comprising: producing trifluoromethyl radicals by radiolysis or photolysis to label a protein of interest for trifluoromethyl radical footprinting.

61. A method of screening possible trifluoromethyl radical precursors, the method comprising:
measuring a scavenging ratio for a potential precursor of interest, thereby identifying a precursor candidate when the scavenging ratio exceeds a predetermined value.
